(19) United States
(12) Patent Application Publication
(10) Pub. No.: US 2018/0050012 A1
(43) Pub. Date: Feb. 22, 2018

(54) Method for Inhibiting Growth of Ovarian Cancer Cells

(71) Applicant: NEW BELLUS ENTERPRISES CO., LTD., Tainan Hsien (TW)

(72) Inventors: CHUN-CHIH HUANG, Pingtung County (TW); YEW-MIN TZENG, Taichung City (TW); CHI-TAI YEH, Taipei City (TW); Tsang-Hsien Alexander Wu, Taipei City (TW)

(73) Assignee: NEW BELLUS ENTERPRISES CO., LTD., Tainan Hsien (TW)

(21) Appl. No.: 15/671,183

(22) Filed: Aug. 8, 2017

(30) Foreign Application Priority Data
Aug. 19, 2016 (TW) TW 105126636

(51) Int. Cl. A61K 31/341; C07D 307/20; A61K 31/122

(52) U.S. Cl. CPC A61K 31/341; C07D 307/20; A61K 2300/00; A61K 2121/00; A61K 31/122

(57) ABSTRACT

The present invention is directed to a method for inhibiting growth of ovarian cancer cells in a subject in need thereof, comprising administering to said subject a composition comprising an effective amount of 4-acetyl-antroquinonol B or a pharmaceutical acceptable salt thereof, and a pharmaceutically acceptable carrier.

### CROSS-REFERENCES TO RELATED APPLICATIONS

The present invention claims foreign priority to Taiwanese patent application No. TW 105126636, filed on Aug. 19, 2016, which is hereby incorporated by reference in its entirety.

### FIELD OF THE INVENTION

The present invention is related to a method of treating ovarian cancer by using 4-acetyl-antroquinonol B to inhibit growth of ovarian cancer cells.

### BACKGROUND OF THE INVENTION

Cancer, a kind of disease, can generally be regarded as a malignant tumor, which is characterized by abnormal clumps of malignant tissue, due to excessive cell division. Cancer cells do not have growth limits as normal cell, so that cancer cells will invade and occupy the space which belongs to normal cell. Types of cancer treatment include chemotherapy, surgery, radiotherapy, and the combinations thereof. Chemotherapy typically involves the use of one or more compounds that inhibit the growth of cancer cells.

Ovarian cancer is the second cause of death among female gynecological cancers. Available treatments for ovarian cancer include debulking surgery or debulking surgery plus chemotherapy, which will differ according to the course of the disease. However, these available treatment options have no therapeutic effect on terminal cancer patients. More than 70% of ovarian cancer patients relapse after chemotherapy and have poor prognosis, while the five-year survival rate is lower than 20%. Therefore, it is very important to develop an effective treating strategy against those resistant ovarian cancer cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the relationship between different concentrations of 4-acetyl-antroquinonol B and the viability of ovarian cancer cell line and protein expression. FIG. 1A: The structure of 4-acetyl-antroquinonol B; FIG. 1B: SRB assay for evaluating cytotoxicity of 4-acetyl-antroquinonol B to different ovarian cancer cell lines (including ovarian cancer cell lines ES-2 and OV-2008); FIG. 1C: Atg5, Atg7, and LC3BII expressions in ES-2 and OV-2008 cell lines; FIG. 1D: fluorescent staining of Atg5, Atg7 and LC3BII in ES-2 and OV-2008 cell lines.

FIG. 2 shows the effects of different concentrations of 4-acetyl-antroquinonol B on autophagy. FIG. 2A: immunohistological staining of LC3BII expression in cell lines treated with 4-acetyl-antroquinonol B and an anti-cancer drug; FIG. 2B: western blotting of LC3BII expression in cell lines treated with 4-acetyl-antroquinonol B and an anti-cancer drug; FIG. 2C: western blotting of Atg7 and Atg5 in cell lines treated with different concentrations of 4-acetyl-antroquinonol B; FIG. 2D: the growth of cell colonies treated with different concentrations of 4-acetyl-antroquinonol B.

FIG. 3 shows the effect of 4-acetyl-antroquinonol B on AKT/mTOR/GSK-3β/p70S6K signal molecules in ES-2 and OV-2008 cell lines with different processing time.

FIG. 4 shows the synergistic effect of 4-acetyl-antroquinonol B and cisplatin. FIG. 4A: the effects of different concentrations of 4-acetyl-antroquinonol B and cisplatin on cell viability; FIG. 4B: combinational index of 4-acetyl-antroquinonol B and cisplatin.

FIG. 5 shows the assessment of anti-cancer efficacy and safety of 4-acetyl-antroquinonol B and an anti-cancer drug in ovarian cancer animal models via oral and intraperitoneal administration. FIG. 5A: effect of oral test on tumor growth; FIG. 5B: effect of intraperitoneal test on tumor growth and photos; FIG. 5C: effect of intraperitoneal test on animal body weight (safety).

FIG. 6 shows different clinical tissue samples from 60 ovarian cancer patients.

### SUMMARY OF THE INVENTION

The present invention is directed to a method for inhibiting growth of ovarian cancer cells in a subject in need thereof, comprising administering to said subject a composition comprising an effective amount of 4-acetyl-antroquinonol B or a pharmaceutical acceptable salt thereof, and a pharmaceutically acceptable carrier.

### DETAILED DESCRIPTION OF THE INVENTION

The present invention provides a method of inhibiting growth of cancer cells, treating, or preventing cancer, especially ovarian cancer, by using a compound of formula I (i.e. 4-acetyl-antroquinonol B) or its pharmaceutically acceptable salt thereof.

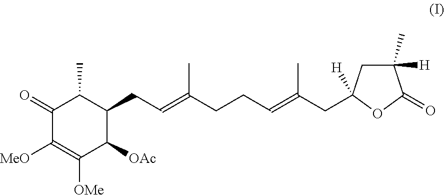 [SMILES: COC1=C(OC)[C@H](OC(C)=O)[C@H](C/C=C(\C)CC/C=C(\C)C[C@H]2C[C@H](C)C(=O)O2)[C@@H](C)C1=O]

More specifically, the present invention provides a pharmaceutical composition for inhibiting growth of ovarian cancer cells, even treating or preventing ovarian cancer, wherein the composition comprises an effective amount of 4-acetyl-antroquinonol B or a pharmaceutical acceptable salt thereof and a pharmaceutically acceptable carrier.

The present invention mainly provides a pharmaceutical composition to inhibit growth of cancer cell. The experiments show that 4-acetyl-antroquinonol B has special significance for four kinds of ovarian cancer cell lines, respectively. ES-2 cell line is derived from a clear cell carcinoma cell line which is highly resistant to chemotherapy drugs (e.g. cisplatin) and has poor prognosis. SKOV-3 and OV-2008 are serous cystadenocarcinoma cell lines, and OV-2008 is significantly responsive to platinum-containing chemotherapeutic drugs. The results of the invention show that all kinds of the ovarian cancer cell lines used herein are responsive to 4-acetyl-antroquinonol B. It is interesting to note that ES-2, the most resistant to cisplatin, has the most pronounced response to 4-acetyl-antroquinonol B, suggesting that there may have a certain degree of relevance between 4-acetyl-antroquinonol B and tumor hyperplasia, and drug resistance. More importantly, 4-acetyl-antroquinonol B has significant effect on inhibiting autophagy, which reduces the expression of autophagy protein Atg-7, leading to the inhibition of downstream Atg-5 expression. Atg-5 plays an important role in autophagosome elongation; therefore, the decrease in Atg-5 expression will reduce the number of mature autophagosomes. The results of the present invention show that 4-acetyl-antroquinonol B can inhibit autophagy by inhibiting autophagosome maturation. In addition, because the reduction in cell autophagy reduces cell viability, the cell colony formation efficiency also decreases after 4-acetyl-antroquinonol B treatment.

The present invention further explores the synergistic effect of 4-acetyl-antroquinonol B and cisplatin by combinational index (CI). CI is a measure of the synergistic (CI<1), additive (CI=1), or antagonistic (CI>1) effect of two or more drugs. The results of the invention show that the combination of 4-acetyl-antroquinonol B and cisplatin provides a better anti-cancer effect. In summary, 4-acetyl-antroquinonol B is a potential chemotherapeutic substance targeting ovarian cancer cells.

The key to overcoming ovarian cancer chemotherapy resistance is to focus on those cells that are resistant to chemotherapy. These cells are characterized by rapid aging, high metabolic demands, and highly activated autophagic-flux. Therefore, regulating autophagy pathways could contribute to the treatment of ovarian cancer. In the present invention, it is found that 4-acetyl-antroquinonol B has an anti-tumor effect on chemotherapy resistant ovarian cancer cells by regulating autophagy-related genes (e.g. Atg-5), and can be administered alone as a monotherapy or in combination with cisplatin as a combination therapy.

Furthermore, the present invention uses ES-2 ovarian cancer cell line to induce tumor growth in NOD-SCID mice, thereby establishing a malignant ovarian cancer animal model for evaluating the efficacy of oral and intraperitoneal administration of 4-acetyl-antroquinonol B for ovarian cancer treatment. In the animal model of ovarian cancer, the ovarian cancer cell line ES-2 is implanted by subcutaneous injection to simulate the clinical symptoms of malignant ovarian cancer. At the same time, the animals are fed with different concentrations of 4-acetyl-antroquinonol B every day for six weeks, and are sacrificed in the first, second, third, fourth, fifth, and sixth week, respectively. The size of the tumors are measured weekly with a vernier caliper, and the changes of tumor size are expressed in ratio. The tumor sizes of the mice administered alone with 4-acetyl-antroquinonol B or cisplatin, and the mice co-administered 4-acetyl-antroquinonol B and cisplatin are all smaller than the tumor sizes of the mice in control group, in which the co-administration of 4-acetyl-antroquinonol B and cisplatin has the best effect in inhibiting tumor growth. The tumor volume of the two experimental groups increases by only about 3 times, while the tumor volume of the control group can grow to about 9 times. As for safety, the body weight changes of each group of mice were monitored weekly. The mice administered alone with cisplatin show sustained decrease in body weight from approximately 26 g to about 21 g. However, there was no significant difference in body weight between the mice co-administered 4-acetyl-antroquinonol B and cisplatin and the mice of control group. Comparing the tumor growth status, administration of either 4-acetyl-antroquinonol B or cisplatin can effectively inhibit cell growth of ovarian cancer cell line ES-2, while co-administration of 4-acetyl-antroquinonol B and cisplatin can prevent excessive weight loss in mice, thus decreases the damage caused by cisplatin to the individual. Finally, the tumor severity of the experimental groups fed with 4-acetyl-antroquinonol B, regardless of oral administration or intraperitoneal injection, is less than that in the control group. It shows that 4-acetyl-antroquinonol B can not only inhibit tumor growth but also have synergistic inhibitory effect with Cisplatin and FOLFOX (folic acid, Fluorouracil, and Oxaliplatin) on ovarian cancer. Therefore, 4-acetyl-antroquinonol B can be a potential adjuvant therapy agent in the treatment of colorectal and ovarian cancer.

In addition, the present invention uses immunohistological staining to analyze the Atg-5-labeled tissue array from ovarian patient (n=60) to investigate the correlation between Atg-5 and disease progress of ovarian cancer. The result shows that 4-acetyl-antroquinonol B has significant cytotoxicity to certain types of ovarian cancer. It is interesting that those cells highly resistant to cisplatin are more responsive to 4-acetyl-antroquinonol B. This is due to high metabolic demands and autophagy of these cells. This phenomenon is also consistent with the previous immunohistological staining result. Atg-5 is usually considered to stimulate ovarian cancer (OR: 5.133; p=0.027). However, 4-acetyl-antroquinonol B can successfully inhibit the expressions of Atg-7 and Atg-5, hence reduce the occurrence of cell autophagy. This effect is similar to an autophagy inhibitor, hydroxychloroquin, which is under clinical trial. 4-acetyl-antroquinonol B not only has the potential of being used as monotherapy but also can be co-administered with cisplatin.

As having one or more chiral center, 4-acetyl-antroquinonol B has various stereoisomeric forms. The 4-acetyl-antroquinonol B mentioned in the present invention includes all such stereoisomers. 4-acetyl-antroquinonol B has the effect of selectively inhibiting the growth of cancer cells. Due to its ultralow molecular weight, a lower dosage of 4-acetyl-antroquinonol B or a pharmaceutically acceptable salt thereof can be used, together with a pharmaceutically acceptable carrier, to receive a desired therapeutic effect.

The present invention is related to a method for inhibiting growth of cancer cells and even treating or preventing cancer in a subject in need thereof (the subject is suffered from a cancer, has a symptom of cancer, or is cancer-prone), by administering to said subject an effective amount of 4-acetyl-antroquinonol B or a pharmaceutically acceptable salt thereof to heal, recover, alleviate, ease, change, treat, improve, or affect the disease, the symptoms of the disease, or the cancer-prone constitution. The term “effective amount” used herein refers to an amount of 4-acetyl-antroquinonol B or a pharmaceutically acceptable salt thereof that can effectively inhibit or treat the disease. The effective amount varies depending on the route of administration, excipient usage, and other co-usage active agents.

The present invention provides a use of a composition containing an effective amount of 4-acetyl-antroquinonol B or a pharmaceutically acceptable salt thereof, and a pharmaceutically acceptable carrier, for preparing pharmaceutical compositions for inhibiting growth of ovarian cancer cells.

The composition of the present invention further comprises an anti-cancer drug including Fluorouracil, Oxaliplatin, or a combination of Fluorouracil and Oxaliplatin.

The present invention also provides a use of a composition containing an effective amount of 4-acetyl-antroquinonol B or a pharmaceutically acceptable salt thereof, an anti-cancer drug, and a pharmaceutically acceptable carrier, for preparing pharmaceutical compositions for inhibiting growth of ovarian cancer cells, wherein the anti-cancer drug includes Fluorouracil, Oxaliplatin, or a combination of Fluorouracil and Oxaliplatin. The composition of the present invention can prevent a subject from weight loss due to anti-cancer drug intake.

In an embodiment, the effective amount of 4-acetyl-antroquinonol B ranges from 0.01 μM to 1000 μM. The concentration of Fluorouracil ranges from 5 mg/mL to 300 mg/mL. The concentration of Oxaliplatin ranges from 0.5 mg/mL to 50 mg/mL.

In another embodiment, the effective amount of 4-acetyl-antroquinonol B ranges from 0.5 μM to 50 μM.

As having one or more chiral center, 4-acetyl-antroquinonol B has various stereoisomeric forms. The 4-acetyl-antroquinonol B mentioned in the present invention includes all such stereoisomers. 4-acetyl-antroquinonol B has the effect of selectively inhibiting the growth of cancer cells. Due to its ultralow molecular weight, a lower dosage of 4-acetyl-antroquinonol B or a pharmaceutically acceptable salt thereof can be used, together with a pharmaceutically acceptable carrier, to receive a desired therapeutic effect.

The present invention is related to a method for inhibiting growth of ovarian cancer cells and even treating or preventing cancer in a subject in need thereof (the subject is suffered from a cancer, has a symptom of cancer, or is cancer-prone), by administering to said subject an effective amount of 4-acetyl-antroquinonol B or a pharmaceutically acceptable salt thereof to heal, recover, alleviate, ease, change, treat, improve, or affect the disease, the symptoms of the disease, or the cancer-prone constitution. The term “effective amount” used herein refers to an amount of 4-acetyl-antroquinonol B or a pharmaceutically acceptable salt thereof that can effectively inhibit or treat the disease. The effective amount varies depending on the route of administration, excipient usage, and other co-usage active agents.

The term “cancer” used herein refers to a cell tumor. Cancer cells have the ability of autonomous growth, which means that the cells can rapidly proliferate under an abnormal state or condition. The term “cancer” used herein includes all kinds of cancerous growth or oncogenic processes, tissue metastasis, malignant transformation of cells, tissue, or organs (unrelated to histopathology), or invasion stage. Examples of cancer include but are not limited to carcinoma and sarcoma, such as breast cancer, leukemia, sarcoma, lymphomas, osteosarcoma, glioma, pheochromocytoma, hepatoma, melanoma, cancer, skin cancer, colorectal cancer, gastric cancer, pancreatic cancer, renal cancer, prostate cancer, testicular cancer, head and neck cancer, brain cancer, esophageal cancer, bladder cancer, adrenal cortical cancer, lung cancer, bronchus cancer, endometrial cancer, nasopharyngeal cancer, cervical cancer, liver cancer, or cancer originated from an unknown position.

4-acetyl-antroquinonol B is prepared through extraction of mycelium of Antrodia cinnamomea (a fungi) with an organic solvent followed by purification via silica gel column chromatography; or is prepared through other chemical synthesis method(s). For example, “extraction of mycelium of Antrodia cinnamomea” refers to the extract of mycelium of Antrodia cinnamomea extracted from the mycelium of Antrodia cinnamomea with appropriate growth. The extract of mycelium of Antrodia cinnamomea is extracted by extraction technique well-known in the art. For example, the dried and ground mycelium of Antrodia cinnamomea may be suspended in a solvent or a mixture of two or more solvents for a sufficiently long time. Examples of suitable solvent include but are not limited to: water, methanol, ethanol, methylene chloride, chloroform, acetone, ether (e.g. diethyl ether), ethyl acetate, and hexane. Then, the solid residue is removed (for example by filter) to get the extract solution of the mycelium of Antrodia cinnamomea, which could be purified through silica gel column chromatography to obtain 4-acetyl-antroquinonol B.

Over the past two decades, natural compounds existing in Antrodia cinnamomea have been studied around the world. In addition to such macromolecules as polysaccharides, 78 small molecule compounds are identified as well, including 31 triterpenoids compounds. Many of these compounds were studied for their pharmacological activity, particularly for anti-cancer activity. A higher dosage of triterpenoids is necessary for achieving the effect of cancer clinical chemotherapy drugs (Geethangili M and Tzeng Y M, Review of pharmacological effects of Antrodia camphorata and its bioactive compounds, Evidence-based Complementary and Alternative Medicine, Aug. 17, 2009; doi: 10.1093/ecam/nep108).

Also, it is found in the present invention that 4-acetyl-antroquinonol B has high inhibitory effect on different ovarian cancer cell lines (ES-2 cell line is derived from a clear cell carcinoma cell line which is highly resistant to chemotherapy drugs (e.g. cisplatin) and has poor prognosis; SKOV-3 and OV-2008 are serous cystadenocarcinoma cell lines). It must be re-emphasized that, among the various natural compounds contained in Antrodia cinnamomea, 4-acetyl-antroquinonol B is one of the few natural compounds proved to have better inhibitory effect on ovarian cancer cell line.

When the composition of the present invention is used in treatment, 4-acetyl-antroquinonol B or a pharmaceutically acceptable salt thereof could be administered simultaneously or separately by way of oral administration, parenteral administration, an inhalation spray, or an implanted reservoir. The term “parenteral administration” used herein refers to subcutaneous, intracutaneous, intravenous, intramuscular, intraarticular, intraarterial, intrasynovial, intrasternal, intrathecal, intraleaional, and intracranial injection and perfusion.

4-acetyl-antroquinonol B and/or pharmaceutically acceptable salts thereof used in the present invention may form an appropriate pharmaceutical form together with at least one solid, liquid or semi-liquid excipient or adjuvant, wherein the pharmaceutical form includes but is not limited to a tablet, capsule, emulsion, aqueous suspension, dispersion, and solution. The carrier generally used in a tablet includes lactose and corn starch. Generally, a lubricating agent, e.g. magnesium stearate, is also added to the tablet. The diluent used in a capsule form include lactose and dried corn starch. When an aqueous suspension or emulsion is used in oral administration, an active ingredient may be suspended or dissolved in an oily phase that binds to the emulsifying or suspending agent. Specific sweetener, flavoring agent, and coloring agent may be added if desired.

The 4-acetyl-antroquinonol B or a pharmaceutically acceptable salt used in the present invention could also be formulated as a sterile injection ingredient (e.g. water or oil suspension), for example by using a suitable dispersing agent or wetter (e.g. Tween 80) and a suspending agent through techniques known in the art. A sterile injection solution or suspension may also be added to a nontoxic parenteral diluent or solvent (e.g. 1,3-Butanediol) to form a sterile injection formulation. Usable vehicles and solvents include mannitol, water, Ringer's solution, and isotonic NaCl solution. In addition, sterile fixed oil is usually used as a solvent or suspension medium (e.g. synthetic monoglyceride or diglycerides). Fatty acid (e.g. oleic acid) and glyceride derivatives thereof may also be used in the preparation of injections; the oil is pharmaceutically acceptable natural oil, e.g. olive oil or castor oil, especially its polyoxyethylated variants. Those oil solution or suspension may also include a long chain alcohol diluent or dispersant, carboxymethyl cellulose, or other similar dispersants.

4-acetyl-antroquinonol B or a pharmaceutically acceptable salt thereof used in the present invention could also be formulated as an inhalation component according to techniques well known in the art. For example, it can be used to make a salt solution by utilizing benzyl alcohol, other suitable preservatives, sorbefacient which can enhance bioavailability, fluorocarbon, or other solubilizing or dispersing agents well known in the art.

The carrier for a pharmaceutical composition must be “acceptable”, which is compatible with the active ingredient of the formulation (preferably having the ability of stabilizing the active ingredient) and is not harmful to the patient. For example, a solubilizing agent (e.g. cyclodextrin), which forms a specific, more soluble complex with one or more active compounds of extracts, is used as an pharmacological adjuvant for delivering active ingredients. Other examples of carriers include colloidal silicon dioxide, magnesium stearate, cellulose, and sodium lauryl sulfate.

Furthermore, since an anti-cancer agent is prone to toxicity when administered to a patient in a high dosage, the pharmaceutical composition of the present invention comprises a safe and effective amount of 4-acetyl-antroquinonol B for inhibiting growth of cancer cells, wherein the safe and effective amount ranges from 0.01-1000 μM, preferably 0.5-50 μM. The specific dose administered to individual patients depends on all possible factors, e.g. the activity of the particular compound used, age, body weight, general health status, gender, dietary status, time and route of the administration, rate of elimination, combination of medical substances, and the severity of the disease to be treated etc.

### EXAMPLES

The following examples are merely illustrative of the present invention. The scope of the present invention is not limited to the following examples. In order that the foregoing and other objects, features and advantages of the present invention will become more apparent, the following preferred embodiments are set forth for detailed description:

### Example 1: The Preparation of 4-Acetyl-Antroquinonol B

3 kg mycelium of Antrodia cinnamomea was heated to reflux and extracted with 10 L of 95% ethanol four times. The extract solution was filtered, concentrated, and dried under reduced pressure to obtain an ethanol extract (384 g). The ethanol extract was suspended in water and partitioned with an equal amount of ethyl acetate. The ethyl acetate layer was concentrated under reduced pressure to obtain an ethyl acetate layer fraction of 157.57 g and an aqueous layer fraction of 159.51 g.

The above 157.57 g ethyl acetate layer fraction was processed by chromatography on a silica gel column (10 cm i.d×30 cm), eluted with n-hexane→n-hexane-ethyl acetate (10:1→10:2→10:3→10:4→10:5→1:1→1:2, v/v)→ethyl acetate→methanol (each 10 L) sequentially, and every 1 L was collected as a fraction. The fraction obtained by elution with n-hexane-ethyl acetate 56-63 (3.015 g) was processed by chromatography on a reverse phase preparative column Tosoh ODS-80 Ts (21.5 mm×300 mm, 10 μm) with H₂O—CH₃CN (20:80) as the mobile phase, a flow rate of 10 ml/min, a detection wavelength of 265 nm, and a column temperature of 40° C. to obtain 4-acetyl-antroquinonol B (131 mg).

### Example 2: Biological Activity Assay

1. The Activation of Frozen Cells:

The principle of activation of frozen cells was rapid thawing to avoid re-crystallization of ice crystals that would cause damage to cells, leading to cell death. After the cell activation, it took about a few days, or one to two generations for letting the cell growth or characteristic performance return to normal, for example, to produce monoclonal antibodies or other proteins. Frozen cells were rapidly thawed by the following description. Frozen tubes were removed from a liquid nitrogen or dry ice container and were immediately placed into a 37° C. tank for quick thawing. The frozen tubes were lightly shaken and the frozen cells were all melted in 3 minutes. The outside of the tubes was wiped with 70% alcohol. Then, the tubes were moved into a sterile laminar flow. The thawed cell suspension was removed and slowly added into a culture container with culture medium (dilution ratio was 1:10˜1:15). The mixture was mixed evenly and incubated in a CO₂ incubator. The next day, the medium was replaced.

2. Culture of Human Cancer Cells:

The ovarian cancer cell lines used in this study included: ES-2, OV-2008, and SKOV-3 obtained from American Type Culture Collection (ATCC). ES-2 was a clear cell carcinoma cell line with high resistance to platinum chemotherapies. Compared with other types of cancer, the clear cell carcinoma was found having poor prognosis in previous studies. SKOV-3 and OV-2008 were more benign cell lines compared to ES-2. Cell culture medium was McCoy5A culture medium (GIbco, 16600-082) supplemented with 1% antibiotic (Gibco, 15140) and 10% fetal bovine serum (FBS, Sigma, F7524). The cells were incubated in a standard incubator (Shel Lab, Sheldon Manufacturing, USA) at 37° C., 5% CO₂.

3. Drug Treatment for Cells:

All the tested cells were incubated in a medium containing 10% fetal bovine serum. When the cells grew to about eighty percent full, the old culture medium was drained and the cells were washed with PBS (phosphate buffered saline) buffer solution. Then, 10 ml of serum-free culture medium was added. Different drugs were added depending on experimental purposes. The reaction was carried out in a 37° C. constant temperature incubator.

4. Cytotoxicity Test:

The ovarian cancer cell lines ES-2, OV-2008, and SKOV-3 were placed in a 96-well culture plate (2000 cells/per well) and incubated overnight in 100 μl of complete DMEM. 50 μl of complete DMEM equivalent sample containing 4-acetyl-antroquinonol B (0.5-50 μM) was added to the different wells of the culture plate. In addition, only 100 μl of complete DMEM was added to the control group. After 2 days of culture, the number of cells in each well was determined by sulforhodamine B (a protein-binding dye). Briefly, the cells were fixed in 10% trichloroacetic acid and stained with 0.4% sulforhodamine B. After stained for 20 minutes, the cells were washed with 1% acetic acid. Thereafter, the cell-bound sulforhodamine B was dissolved in 10 mM Tris base. The absorbance (optical density) was determined at 562 nm by a microtiter plate reader.

5. Clonogenicity Analysis

In a 6-well plate was seeded 600 cells per well. The culture medium was McCoy5A supplemented with 10% fetal bovine serum.

6. Western Blot

Western blot is carried out in a standard procedure. The cells were washed twice with PBS and then a cell lysis solution was added. Cell debris was removed by centrifugation to collect protein degradation products. The protein concentrate was quantified by BCA assay kit (Pierce, Thermo Scientific, USA). Each sample was taken an equal amount of protein degradation products to 10% SDS-PAGE electrophoresis then transferred to a PVDF membrane, followed by the addition of anti-autophagy-related genes (Atg-7, Atg-5, and LC3BII) primary antibodies, and an anti-β-actin antibody as a control group. After the target proteins and the primary antibodies reacted overnight, the secondary antibody conjugated to HRP was added. Finally, the signals of the target proteins were determined by a luminescence-based imaging system (UVP, LLC, USA).

7. Analysis of Drug Combinational Index (CI)

The combined effects of two drugs by Chou-Talalay algorithm and Compusyn software (ComboSyn Incorporated, Paramus, N.J., USA) are analyzed to explore the drug combination effect of 4-AAQB and cisplatin. CI is an indicator of the synergistic (CI<1), additive (CI=1), or antagonistic (CI>1) effect of two drugs.

### Example 3 Results of Biological Activity Assay

1. 4-Acetyl-Antroquinonol B Inhibited the Growth of Different Ovarian Cancer Cells:

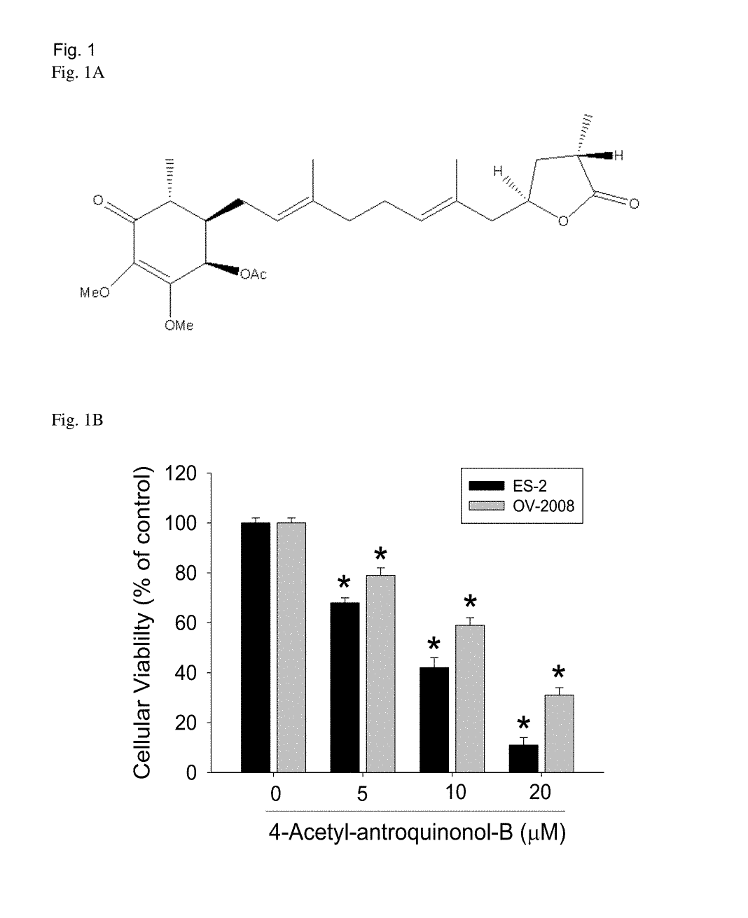

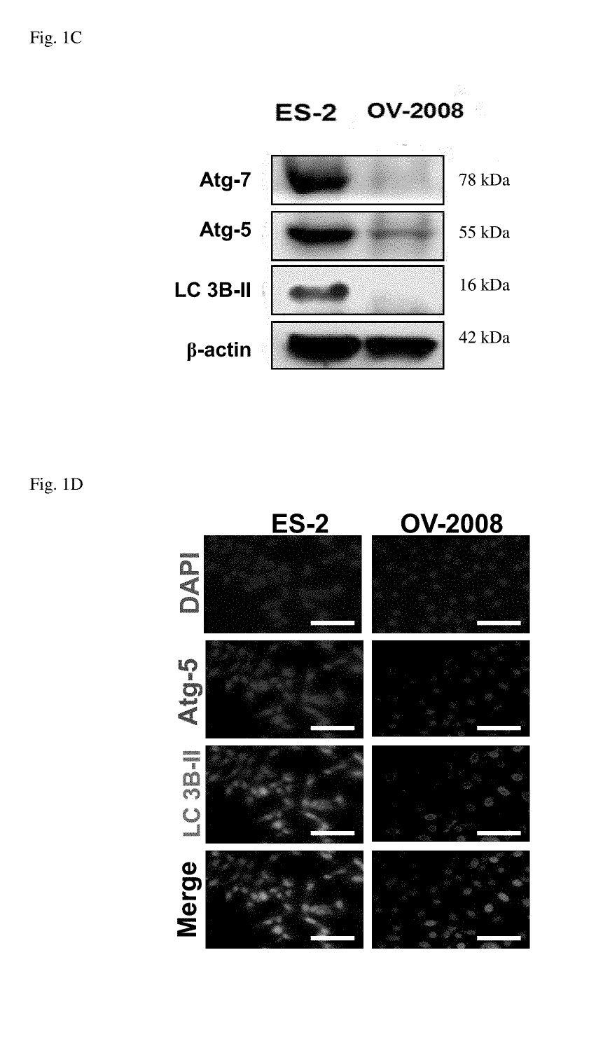

The cytotoxicity of 4-acetyl-antroquinonol B (FIG. 1A) to two different ovarian cancers was first tested by cell viability test. The two ovarian cancer cell lines had special significance, respectively. ES-2 was derived from an ovarian cancer cell line which was highly resistant to chemotherapy drugs (e.g. cisplatin) and has poor prognosis. OV-2008 was a serous cystadenocarcinoma cell line. The experimental results showed that all types of ovarian cancer cell lines used in the present invention were responsive to 4-acetyl-antroquinonol B. Interestingly, the cell line ES-2, the most resistant to cisplatin, had the most pronounced response to 4-acetyl-antroquinonol B (FIG. 1B), suggesting that there might have a certain degree of relevance between 4-acetyl-antroquinonol B and tumor hyperplasia, and drug resistance.

2. Negative Correlation Between Drug Resistance and Cell Autophagy Development:

The level of autophagy basal expression was positively correlated with the resistance to chemotherapies. Anti-tumor therapies including chemotherapy and radiotherapy had been proved to trigger cell autophagy and activate molecular mechanism that enhanced cell viability. Compared with OV-2008, we found that the cell line ES-2 highly resistant to cisplatin had higher Atg5, Atg7, and LC3BII expression level (FIG. 1C). It was also found that Atg-5 and LC3BII were both expressed a lot in highly malignant ES-2 ovarian cancer cells by using cell fluorescence staining. It was speculated that LC3BII was located on the membrane of autophagosomes after phospholipidization. Therefore, the high expression level of Atg-5 and LC3BII also represented the strong performance of autophagy (FIG. 1D).

3. 4-Acetyl-Antroquinonol B Inhibited the Development of Autophagy by Inhibiting Autophagosome Elongation:

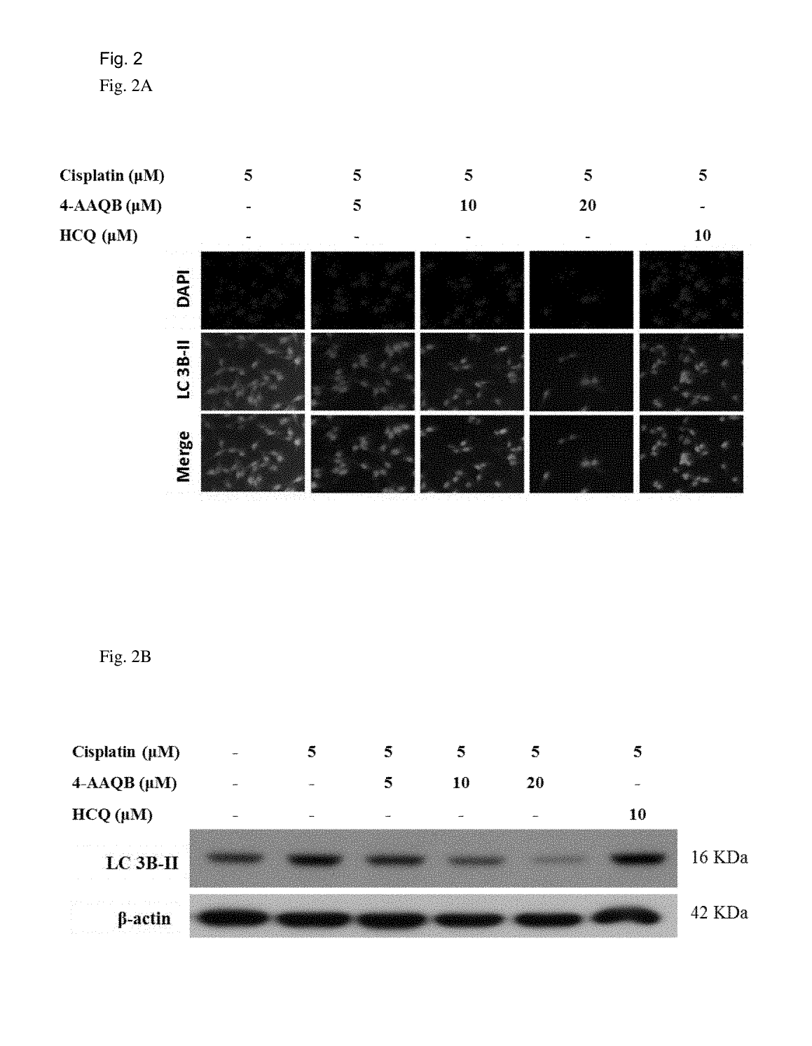

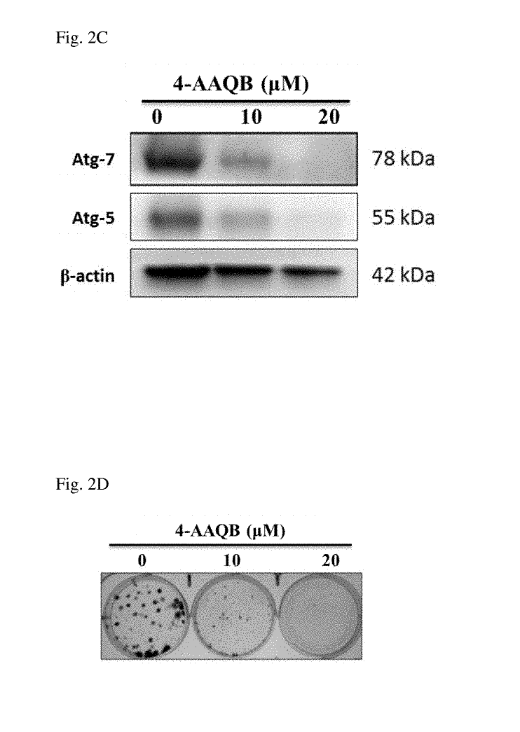

Even if the cell line ES-2 itself had a higher autophagy, the autophagy phenomenon was more significant after cisplatin treatment. Presumably because cisplatin treatment initiated autophagy that enhanced viability. The results showed that 4-acetyl-antroquinonol B significantly inhibited cell autophagy. After 4-acetyl-antroquinonol B treatment, LC3BII was detected by immunohistochemical staining and western blot, showing that 4-acetyl-antroquinonol B did inhibit the LC3BII expression on autophagosomes (FIG. 2A). Cell autophagy would protect cancer cells from apoptosis caused by chemotherapies. Hydroxychloroquine was a widely used antiparasitic drug. The recent phase II clinical study had also confirmed that hydroxychloroquine could successfully inhibit cell autophagy. Hydroxychloroquine increased the pH inside lysosomes thus made the lysosomes difficult to fuse with mature autophagosomes. The Inhibitory effect on autophagy could also solve the problem of cell autophagy resistance due to chemotherapies in cancer cells. In this embodiment, the ES-2 cell line was pretreated with 5 μM cisplatin and then treated with 4-acetyl-antroquinonol B. The results showed that the autophagosome maturation in cells treated with 4-acetyl-antroquinonol B was inhibited more than in cells treated with hydroxychloroquine (FIG. 2B). The cell viability in cells treated with 4-acetyl-antroquinonol B was also significantly lower than that in cells treated with hydroxychloroquine at the same concentration. Based on the above results, the ability of 4-acetyl-antroquinonol B to reduce cell autophagy by inhibiting autophagosome elongation was comparable to the widely used cell autophagy inhibitors. More importantly, according to the results from western blot it was found that mechanism of 4-acetyl-antroquinonol B was different from hydroxychloroquine. Hydroxychloroquine increased the pH inside lysosomes, thereby reducing the fusion of autophagosomes and lysosomes to achieve the effect of inhibiting cell autophagy, while 4-acetyl-antroquinonol B reduced the expression of Atg-7 and thus the expression of downstream Atg-5 was also inhibited (FIG. 2C). Atg-5 played an important role in autophagosome elongation, hence, the decrease in Atg-5 expression would reduce the number of mature autophagosomes. The results showed that 4-acetyl-antroquinonol B achieved the inhibition of autophagy by inhibiting the maturation of autophagosomes. In addition, because the reduction in cell autophagy reduced cell viability, the cell colony formation efficiency also decreased after 4-acetyl-antroquinonol B treatment (FIG. 2D).

4. 4-Acetyl-Antroquinonol B Inhibited ES2 Cell Proliferation and Autophagy by Blocking Signaling Pathway Molecules AKT/mTOR/P70S6K:

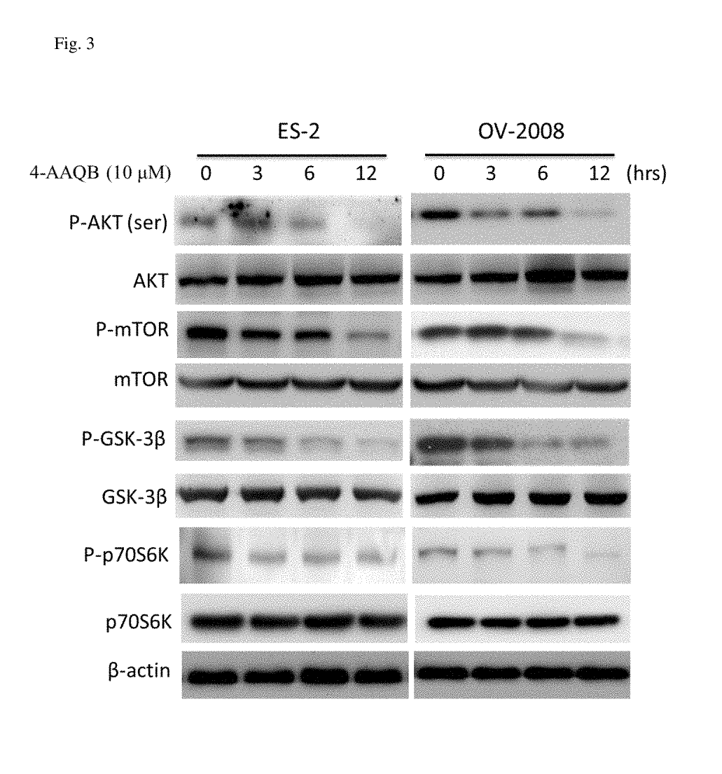

The effect of 4-acetyl-arbutin-B on cell proliferation and autophagy of ovarian cancer cell line ES2 was observed, and the expression of autophagy-associated protein LC3BII and signaling pathway molecules AKT/mTOR/p70S6K were detected by western blot. The experimental results showed that 20 uM 4-acetyl-antroquinonol B significantly inhibited the growth of ES2 cells in a time-dependent effect (P<0.05). The expressions of signaling pathway key molecules AKT/mTOR/p70S6K in ES2 cells were significantly reduced by treatment of 4-acetyl-antroquinonol B for different time length (0, 3, 6, and 12 hours) (FIG. 3).

5. The Combination of 4-Acetyl-Antroquinonol B and Cisplatin had a Better Anti-Cancer Effect:

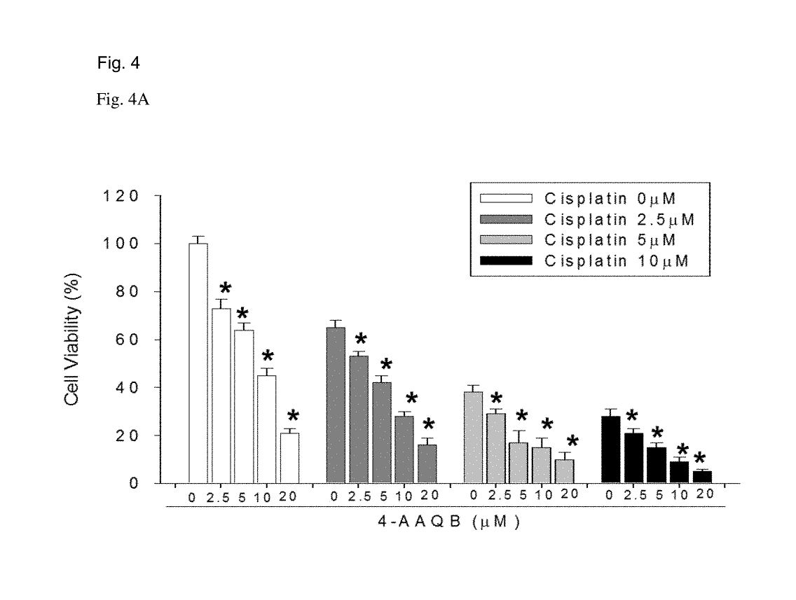

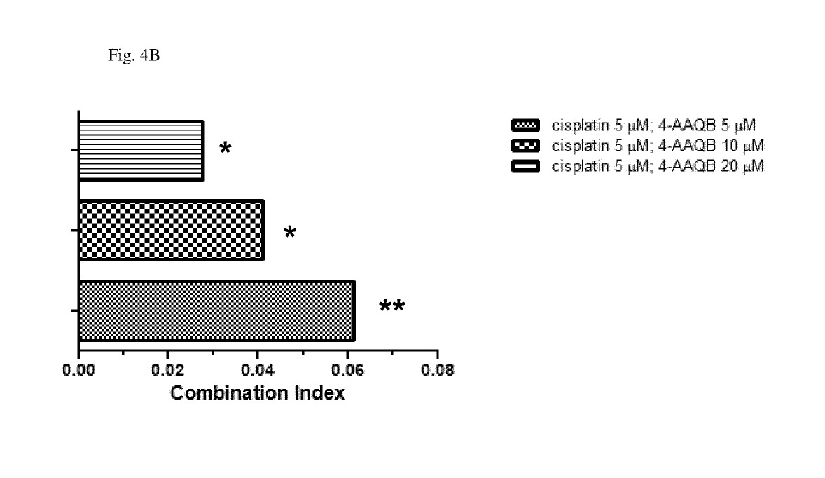

The synergistic effect of 4-acetyl-antroquinonol B and cisplatin was explored by combinational index (CI) in this embodiment (FIG. 4A). The results showed that different concentrations of 4-acetyl-antroquinonol B (5, 10, and 20 μM) had synergistic effect with cisplatin (5 μM) (FIG. 4B). According to the above, 4-acetyl-antroquinonol B inhibited ES-2 cell proliferation by down-regulating the AKT/mTOR/p70S6K signaling pathway, and induced ES-2 autophagy. The combination of 4-acetyl-antroquinonol B and cisplatin enhanced the anti-tumor effect of 4-acetyl-antroquinonol B (FIG. 4B).

6. The Anti-Cancer Effect of 4-Acetyl-Antroquinonol B in Ovarian Cancer Animal Models Via Oral and Intraperitoneal Administration:

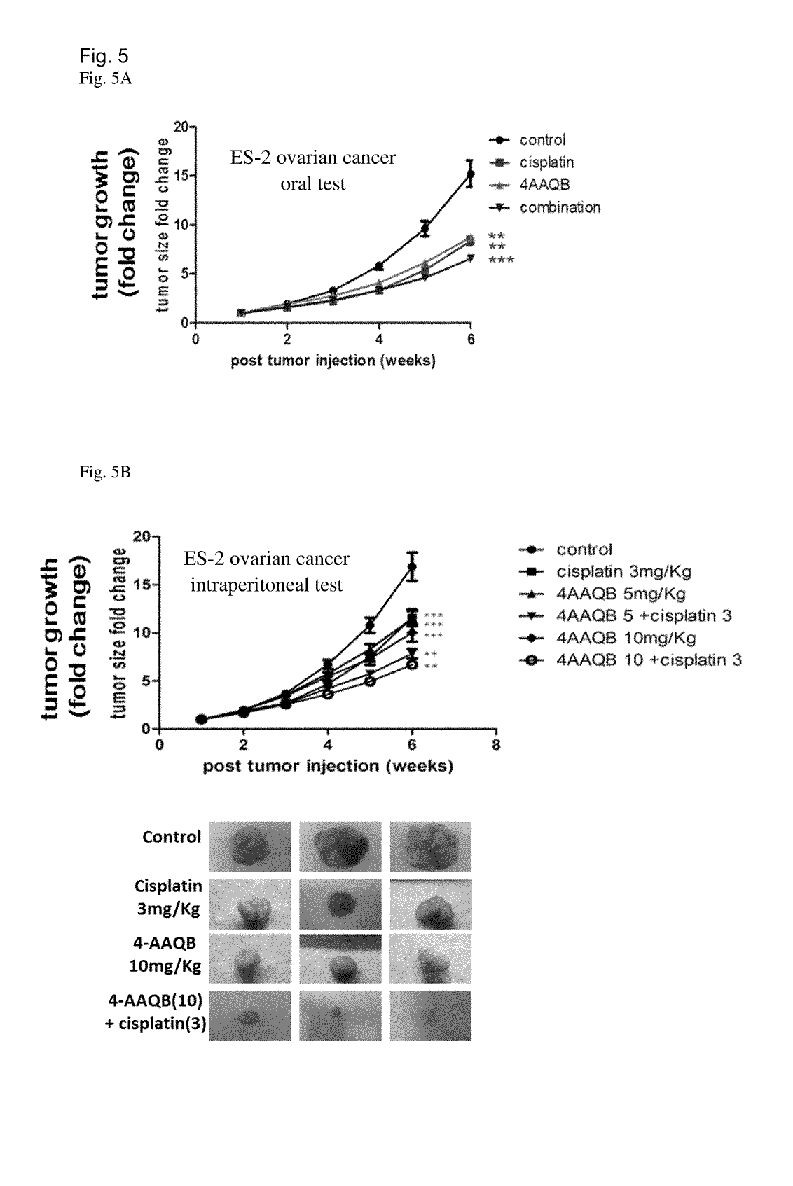

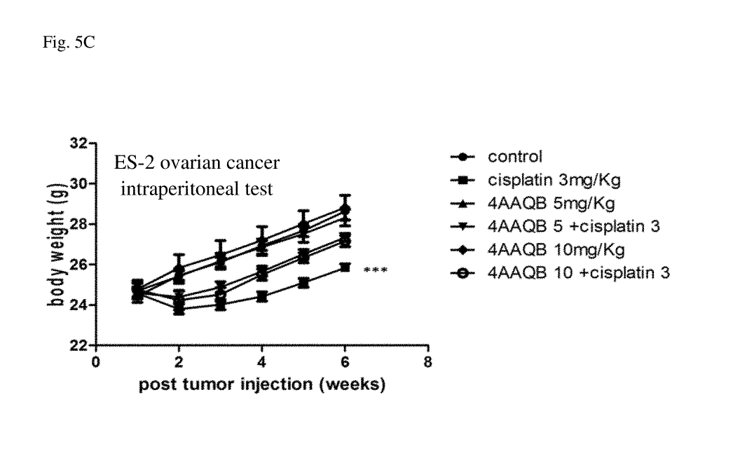

In this study, tumors were induced by malignant ovarian cancer cell line ES-2 in NOD-SCID mice to establish malignant ovarian cancer animal models. The efficacy of oral and intraperitoneal administration of 4-acetyl-antroquinonol B for ovarian cancer was evaluated. In the ovarian cancer animal models, the malignant ovarian cancer cell line ES-2 was implanted subcutaneously to simulate the symptoms of malignant ovarian cancer. And the same time, the animals were fed with different concentrations of 4-acetyl-antroquinonol B every day for six weeks. The animals were sacrificed each week in the six weeks, respectively. The results showed that in the ovarian cancer animal models whether administered orally or by intraperitoneal injection, the tumor severity was significantly lower in the experimental group fed with 4-acetyl-antroquinonol B than in the control group (FIG. 5B). In addition, different doses of 4-acetyl-antroquinonol B were administered orally (5 mg/kg and 10 mg/kg), and 1 dose (3 mg/kg) of cisplatin was injected intravenously each week (three doses total) when the tumor size was about 70-250 mm³. The results showed that 4-acetyl-antroquinonol B administered alone had a dose-dependent inhibitory effect on tumor growth (FIG. 5C). 4-acetyl-antroquinonol B also had synergistic inhibitory effect with cisplatin and FOLFOX on ovarian cancer. Importantly, as for safety of 4-acetyl-antroquinonol B, the body weight changes of each group of mice were monitored weekly. The mice administered alone with cisplatin showed sustained decrease in body weight from approximately 26 g to about 21 g. However, there was no significant difference in body weight between the mice co-administered 4-acetyl-antroquinonol B and cisplatin and the mice of control group. Compare the tumor growth status, administration of either 4-acetyl-antroquinonol B or cisplatin effectively inhibited cell growth of ovarian cancer cell line ES-2, while co-administration of 4-acetyl-antroquinonol B and cisplatin prevented excessive weight loss in mice, thus decreased the damage caused by cisplatin to the individual (FIG. 5D). This result indicated that the safety of 4-acetyl-antroquinonol B in the living body is extremely high. Therefore, 4-acetyl-antroquinonol B was a potential adjuvant therapy agent in the treatment of ovarian cancer.

7. Atg-5 was Associated with Ovarian Cancer Prognosis:

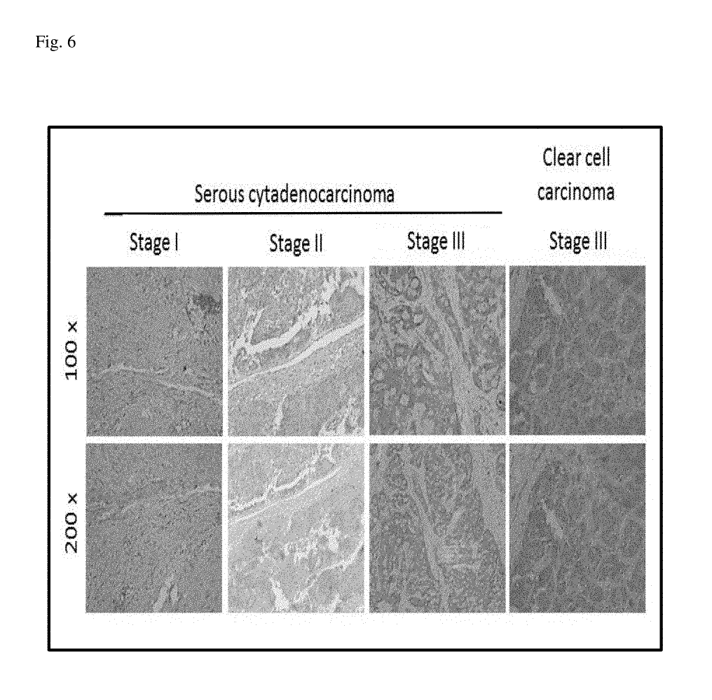

In this example, the relationship between Atg-5 expression and clinical index was studied with tissue samples from 60 ovarian cancer patients. Results of ovarian cancer immunohistochemical (IHC) staining from different clinical classification were integrated in FIG. 6. The results of IHC staining were classified into three groups according to the previous experimental method: no staining (n=9), weak or focused staining (n=30), medium or strong staining (n=21). The results showed that Atg-5 was expressed higher in malignant tumor cells, and the expression was positively correlated with disease progress. Atg-5 staining was higher in tissues of terminal cancer patients than in tissues of early cancer patients. In addition, patient survival rate in the group of medium or strong Atg-5 staining was significantly lower than that in both groups of no staining and weak or focused staining (OR: 5.133; p=0.027). Therefore, Atg-5 had potential to become a pathological target of ovarian cancer prognostic indicator.

### Example 4: Animal Test Method

Experimental Animals:

Immunodeficient mice (NOD/SCID mice, about 4-6 weeks old) were purchased from BioLASCO Taiwan Co., Ltd. The test was initiated after a week of domestication.

Cell Culture:

The selected tumor cells were ES2 malignant ovarian cancer cells. ES-2 cell line was derived from a ovarian cancer cell line which was highly resistant to chemotherapy drugs (e.g. cisplatin) and had poor prognosis. It was an anchorage-dependent cell line with strong transfer capacity. The culture medium was DMEM containing 10% fetal bovine serum (FBS), 1% non-essential amino acids (NEAA), and 1% antibiotics. The cells were incubated in a incubator at 37° C., 5% CO₂ and subcultured once every 3 to 4 days.

The cells were treated with 0.05% trypsin-EDTA for 3 to 5 minutes to be suspended. The serum-containing medium was added to neutralize trypsin. The mixture was then centrifuged at 1000 rpm at 20° C. for 5 min. The supernatant was removed and the cell pellet was gently dispersed. The cells were resuspended in a suitable volume of culture medium. After mixing evenly, a little cell fluid was taken and cells were counted with a cytometer. The cells were diluted to a concentration of 10⁷ cells per milliliter, and approximately 0.15 ml was dispensed into a 1.5 ml small centrifuge tube.

Drug Preparation:

A solution of 4-acetyl-antroquinonol B was prepared in DMSO (250 mg/ml, DMSO was used as the solvent). After 4-acetyl-antroquinonol B was completely dissolved, the solution was dispensed as stock solution and stored at 4° C. Sterile normal saline solution was added to the stock to make a 500-fold dilution. The mixture was mixed evenly and then injected intraperitoneally. Cisplatin, a current clinical standard chemotherapy drug, was also an injection with a concentration of 50 mg/ml or 5 mg/ml, and both of which were injected intravenously without further dilution.

Tumor Cells Injection:

The day before tumor cells injection, zoletil 50 (10-fold dilution) and rompun 2% were mixed (1:1), and 0.25 ml of the mixture was injected intraperitoneally to each mouse for anesthesia. The mice were exposed to radiation to suppress their immunity after they were asleep. The irradiation dose was 0.75 Gy.

Mice were anesthetized with 2.5% isoflurane. The hair of injection site was shaved. The injection site was disinfected with 75% alcohol and povidone-iodine before injection. ES2 tumor cells were injected via 29G insulin needle. At the time of injection, the mouse epidermis was pulled up with tweezers and the ES2 tumor cells were injected subcutaneously. The number of cells injected was 10⁶ and the volume was 0.1 ml. After the injection, the cell fluid was confirmed without leakage, then the mice were moved back into cages for waiting to wake up. The body temperature of the mice was maintained. The tumor growth was continuously observed.

Establishment of ovarian cancer animal models for oral and intraperitoneal administration:

The ES-2 ovarian cancer cells in logarithmic phase were mixed with serum suspension and 0.1 ml (2×10⁶ cells) was inoculated into hind legs of NOD-SCID immunodeficient mice to form tumors. After 48 hours, the mice were randomly divided into control group, cisplatin positive control group (3 mg/kg body weight), high dose group (10 mg/kg body weight), and low dose group (5 mg/kg body weight). There were 6 mice in each group. The in vivo efficacy of 4-AAQB alone or in combination with cisplatin on tumor growth was investigated using ES-2 xenograft models. Changes in tumor volume and body weight were monitored and recorded every week. NOD-SCID mice inoculated with ES-2 cells were then treated with 5 or 10 mg/kg 4-AAQB and/or 3 mg/kg cisplatin daily for 6 weeks, while the control group was treated with the same amount of normal saline solution. The longest diameter (a) and the shortest diameter (b) of the tumor on mice were measured. The tumor size=(a*b2)/2, and the tumor growth curve was calculated.

### Example 5 Results of the Animal Test

Tumor Size Determination:

The tumor size was measured weekly by measuring the longest diameter and the shortest diameter of the tumor with a vernier caliper. To ensure the accuracy of the measurement, the tumor size was measured by the same person during the experiment. The formula for tumor volume calculation: tumor size=(a*b²)/2 (a: the longest diameter; b: the shortest diameter). Finally, the mice were sacrificed and the tumor tissues were taken for taking photos which were saved as files, followed by fixation with formalin. The fold change in tumor volume was calculated and shown in the figure. The fold change in tumor volume=tumor volume (N)/tumor volume (N−1), in which N represented the number of weeks.

What is claimed is:

1. A method for inhibiting growth of ovarian cancer cells in a subject in need thereof, comprising administering to said subject a composition comprising an effective amount of 4-acetyl-antroquinonol B or a pharmaceutical acceptable salt thereof, and a pharmaceutically acceptable carrier.

2. The method of claim 1, wherein the composition further comprises an anti-cancer drug.

3. The method of claim 2, the anti-cancer drug comprises Fluorouracil, Oxaliplatin, or a combination of Fluorouracil and Oxaliplatin.

4. The method of claim 1, wherein the composition has the ability of treating or preventing cancer.

5. The method of claim 1, wherein the 4-acetyl-antroquinonol B is prepared through extraction of mycelium of Antrodia cinnamomea with an organic solvent followed by purification via silica gel column chromatography.

6. The method of claim 1, wherein the effective amount of 4-acetyl-antroquinonol B is 0.01-1000 μM.

7. The method of claim 1, wherein the effective amount of 4-acetyl-antroquinonol B is 0.5-50 μM.

8. The method of claim 3, wherein the amount of Fluorouracil is 5-300 mg/mL.

9. The method of claim 3, wherein the amount of Oxaliplatin is 0.5-50 mg/mL.

10. The method of claim 3, wherein the composition prevents the subject from weight loss due to anti-cancer drug intake.
